(19) United States
(12) Patent Application Publication
(10) Pub. No.: US 2025/0059119 A1
(43) Pub. Date: Feb. 20, 2025

(54) A PROCESS AND AN APPARATUS FOR PRODUCING METHANOL FROM BLACK LIQUOR

(71) Applicant: ANDRITZ OY, Helsinki (FI)

(72) Inventors: Naveen CHENNA, Helsinki (FI); Otto GREIS, Helsinki (FI); Aino PESOLA, Helsinki (FI); Pekka TERVOLA, Helsinki (FI)

(21) Appl. No.: 18/710,936

(22) PCT Filed: Nov. 16, 2022
(86) PCT No.: PCT/FI2022/050754

§ 371 Date: May 16, 2024

(30) Foreign Application Priority Data
Nov. 18, 2021 (FI) 20216183

(51) Int. Cl. C07C 29/50

(52) U.S. Cl. CPC C07C 29/50

(57) ABSTRACT

A process, and a system configured to perform the process, is provided wherein lignin is oxidized into methanol by feeding into a black liquor containing feedstock an oxidative agent in a reactor. The process includes: providing a feedstock comprising black liquor; preconditioning the feedstock in a preconditioning reactor; transferring the feedstock or the preconditioned feedstock to a second reactor; and feeding into the second reactor a first oxidative agent to produce a reaction mixture to oxidize methoxyl groups of lignin present in the black liquor into methanol.

### TECHNICAL FIELD

The present disclosure generally relates to production of methanol. The disclosure relates particularly, though not exclusively, to a process and apparatus for producing methanol from black liquor produced in pulp mills.

### BACKGROUND

This section illustrates useful background information without admission of any technique described herein representative of the state of the art.

Black liquor (BL) produced in pulp mills contains various organic and inorganic compounds. BL contains for example lignin, and from the total amount of dry solids, 13% is phenolic lignin and 19% is non-phenolic lignin.

It is an object to develop a process which uses lignin present in black liquor to produce methanol.

### SUMMARY

The appended claims define the scope of protection. Any example and/or technical description of an apparatus, system, product and/or process in the description and/or drawing which is not covered by the claims, is presented herein not as an embodiment of the invention but as background art or example useful for understanding the invention.

The present process uses a black liquor oxidation reactor typically not present in pulp mills to convert phenolic and/or non-phenolic methoxyl groups of BL lignin to methanol. In an embodiment the present process and/or system is integrated into a pulp mill.

The present operation conditions of the reactor can convert almost all phenolic or/and non-phenolic lignin methoxyl groups to methanol and oxidized lignin. As methanol boils at low temperature, most of the methanol can be extracted by using a simple stripping and/or a distillation column.

It is an object of the present disclosure to provide a process for producing methanol from lignin in waste streams and side streams originating from industrial processes, such as from black liquor. Another object is to provide an alternative solution to existing technology in methanol production and/or in lignin processing.

According to a first aspect is provided process for producing methanol comprising:
i. providing a feedstock comprising black liquor;
ii. optionally preconditioning the feedstock in a preconditioning reactor to provide preconditioned feedstock;
iii. transferring the feedstock or the preconditioned feedstock to a second reactor; and
iv. feeding into the second reactor at least one oxidative agent to produce a reaction mixture to oxidize methoxyl groups of lignin present in the black liquor into methanol.

In the present disclosure a process according to the first aspect, in which the preconditioning step is used, is also referred to as a two-step process or a two-reactor process.

In the present disclosure a process according to the first aspect in which no preconditioning is used, is also referred to as a one-step process or a one-reactor process.

In an embodiment the process comprises preconditioning in alkaline conditions at a temperature in the range 140-300° C. Including the preconditioning step in the process is advantageous for example when using black liquor with high sulfur content. With the preconditioning at least partial removal of sulfuric compounds can be achieved.

In an embodiment the process preconditioning, in which an oxidative agent selected from air, oxygen, and their mixture, is fed into the preconditioning reactor.

In an embodiment in the process the oxidative agent in step iv. is selected from oxygen, ozone, air, or any combination thereof.

In an embodiment, in the process the oxidative agent is fed into the preconditioning reactor and/or to the second reactor through a nozzle or an inlet port.

In an embodiment, in the process the pH of the reaction mixture is adjusted to a value selected from the range 8-14, preferably from the range 9-13, more preferably from the range 11-12.

In an embodiment, in the process the preconditioning reactor, and/or the second reactor, is operated at a temperature in the range 80-160° C., preferably in the range 85-140° C., more preferably at about 90° C.

In an embodiment in the process the preconditioning reactor and/or the second reactor, is operated at a pressure selected from the range 2-16 bar (g), preferably from the range 3-14 bar(g), most preferably form the range 4-10 bar(g), or the pressure is about 8 bar(g).

In an embodiment in the process the feedstock comprises softwood black liquor and/or hardwood black liquor.

In an embodiment in the process the feedstock contains sulfuric compounds.

In an embodiment in the process the oxidized feedstock is transferred back to black liquor circulation of a pulp mill.

In an embodiment in the process the preconditioning reactor, or the second reactor, is operated in conditions wherein methanol is at least partially in gaseous form, and wherein the reactor is directly in fluid connection to a liquefication unit or to a stripper off gas (SOG) line configured to condense methanol. In an embodiment the stripper off gases are collected together and then condensed in a condenser unit.

According to a second aspect is provided a system comprising means for performing the process of the first aspect or any of its embodiment.

In an embodiment of the first aspect the oxidative agent used in the step iv. is different from the oxidative agent used in the preconditioning step.

In an embodiment of the first aspect the oxidative agent in the preconditioning step is air, and the oxidative agent in step iv is selected from oxygen, ozone, air, or any combination thereof.

When the oxidative agent(s) is fed into the feedstock in the present process, a reaction mixture is formed wherein the oxidative agent converts methoxyl groups present in lignin into methanol in oxidative reactions. If more reactive sulfuric compounds are present in the feedstock, as is usually the case when processing black liquor with the present process, the sulfuric compounds are oxidized before lignin oxidation begins. In an embodiment both phenolic and non-phenolic lignin is oxidized by the present process.

In an embodiment the preconditioning reactor, and/or the second reactor is in fluid connection to black liquor pipeline of a pulp mill. These reactors, as well as the other parts of the system disclosed herein, can thus be installed to be an integrated unit of a pulp mill.

In an embodiment oxidized feedstock produced in the reactor is transferred back to a black liquor pipeline of a pulp mill after the oxidative treatment is finished.

In another embodiment the reactor(s) is operated in conditions wherein methanol is at least partially in vapor form, and wherein the reactor is directly in fluid connection to a liquefication unit or to a stripper off gas line, which is used to recover methanol in liquid form.

### BRIEF DESCRIPTION OF THE FIGURES

Some example embodiments will be described with reference to the accompanying figures, in which:

FIG. 1 schematically shows as an example embodiment certain parts of a system configured to carry out the present process in a system comprising two reactors.

FIG. 2 schematically shows as an example embodiment certain parts of a system configured to carry out the present process in a system comprising one reactor.

### DETAILED DESCRIPTION

In the present description like reference signs denote like elements or steps.

In the present disclosure Adt refers to air dry ton.

In an embodiment black liquor is or comprises weak black liquor.

In an embodiment the present process is carried out without adding a catalyst to the reactor.

In an embodiment the preconditioning comprises heating the feedstock preferably in alkaline conditions. The preconditioning step raises the dry solids (DS) contents of the feedstock and simultaneously reduces sulfur content of the feedstock by producing volatile sulfur compounds. Further, when processing kraft black liquor, heat treatment reduces viscosity. The cause for the sulfur release in the heating step (liquor heat treatment, LHT) is the formation of organic sulfur compounds. The amount of methyl mercaptan (MM), dimethyl sulfide (DMS) and dimethyl disulfide (DMDS) are dependent on the sulfidity of the cooking liquor and on the wood species used to produce black liquor. In LHT, the main compounds that are formed and released from the feedstock are MM and DMS. The sulfur compounds formed during LHT preconditioning can be removed as vapors from the preconditioning reactor. When using a LHT reactor as the preconditioning reactor, the LHT reactor can be operated in a temperature range 140-300° C. Preferably the temperature is above 175° C. to achieve better sulfur removal.

In an embodiment the sulfur release is approximate 2-5 kgS/Adt when using black liquor as the feedstock. The sulfuric compounds can be removed from the reactor as vapors, which then continue their way into non-condensable gas (NCG) collection.

Thus, the optional preconditioning step can be used to reduce sulfur dioxide emissions. Further, in the present process the preconditioning step is advantageous because it removes sulfur which would otherwise react with the oxidative agent in the oxidative step in the second reactor. As a result of the preconditioning, the sulfur content of the feedstock is low when contacted with the oxidative agent, and the consumption of the oxidative agent is therefore reduced. When using preconditioned feedstock, the oxidative agent reacts with lignin and not with sulfuric compounds.

When the preconditioning step is used, the feedstock obtained after the preconditioning is herein called preconditioned feedstock.

In an embodiment the process does not comprise the preconditioning step. For example, when the sulfur content of the feedstock is low, the preconditioning step can be omitted. Also, when it is not desirable to reduce the consumption of the oxidative agent, the preconditioning step can be omitted.

In an embodiment the preconditioning comprises oxidizing the feedstock with an oxidative agent. In an embodiment the oxidative agent used in the preconditioning step is air, oxygen, or any mixture thereof.

In an embodiment of the preconditioning the feedstock is heated before adding the oxidative agent. In another embodiment the feedstock is hated to a temperature about 10-20° C. lower than the selected preconditioning temperature before adding the oxidative agent.

In an embodiment the preconditioning is carried out in the same operating conditions as the oxidative treatment in the second reactor.

In an embodiment the preconditioning comprises heating and oxidizing the feedstock with an oxidative agent. In an embodiment the black liquor used in the present process contains 20-50 mass-% dry solids, such as 20, 30, 40 or 50 mass-% dry solids.

In the present disclosure COD refers to chemical oxygen demand expressed as mg/l. The COD can be determined according to ISO 6060:1989 Water quality-Determination of the chemical oxygen demand.

As used herein, the term “comprising” includes the broader meanings of “including”, “containing”, and “comprehending”, as well as the narrower expressions “consisting of” and “consisting only of”.

In an embodiment the process steps are carried out in the sequence identified in any aspect, embodiment, or claim. In another embodiment any process step specified to be carried out to a product or an intermediate obtained in a preceding process step is carried out directly to said product or intermediate, i.e. without additional, optional or auxiliary processing steps that may chemically and/or physically alter the product or intermediate between said two consecutive steps.

In an embodiment the present process is an industrial process. In another embodiment the industrial process may exclude small scale methods such as laboratory scale methods that are not scaled up to volumes used in industry.

In an embodiment lignin present in the black liquor is at least partially dissolved or solubilized.

In the present process, reactive sulfides are at least partially converted to non-reactive sulphates in the beginning of oxidation, or during the preconditioning step. After reactive sulfur is oxidized or otherwise removed from the feedstock, lignin oxidation is carried out either in the same reactor or in a separate reactor, such as a second reactor, to which the desulfurized feedstock is transferred. The lignin oxidation process can be executed with minimal number of reactors in a series, or even in a single reactor where both sulfur oxidation and lignin oxidation take place. When using a single reactor system, the feedstock can be preconditioned by heating and the oxidative agent can be added to a feedstock which has about 10-20° C. lower temperature than the temperature of the oxidative treatment. Use of more than one reactor may be preferable to allow better control of the process, and to use more expensive oxidating agent such as oxygen or ozone only for a feedstock from which reactive sulfur compounds have already been oxidized.

Lignin contains methoxyl groups that can be converted to methanol by the present process.

Lignin is either nonphenolic or phenolic, and approximately 40% of lignin is phenolic and 60% is non-phenolic. Phenolic lignin is reactive to mild oxidative conditions whereas non-phenolic lignin requires more oxidative conditions and more intense environment to produce methanol. With the operating conditions and the oxidative agent disclosed herein, methoxyl groups of phenolic and non-phenolic lignin available in the lignin structure can be converted to methanol.

In the present process the oxidation reactions, and in particular the lignin oxidation reactions, can be executed in at least one oxidation reactor, such as a second reactor, to convert the phenolic or/and non-phenolic methoxyl groups of lignin into methanol. In another embodiment more than one, such as two, three or four, lignin oxidation reactors in a series are used. Thereby, instead of a single second reactor, the lignin oxidation can be carried out in a reaction zone comprising a plurality of lignin oxidation reactors.

In an embodiment the pressure and temperature inside at least one reactor are selected such that methanol remains in liquid phase.

In an embodiment pH of the reaction mixture is adjusted to a value selected from the range 8-14, preferably in the range 12-14. The adjustment can be made with any alkali or acid. In an embodiment the pH adjustment is made with an alkali selected from sodium hydroxide, white liquor and oxidized white liquor. The pH adjustment can be made initially at the beginning of the oxidative treatment, and/or during the lignin oxidation reaction to keep the pH at or near the selected pH value. Optionally the pH is adjusted to the above value when carrying out oxidation of the sulfur compounds.

In an embodiment the reactor is operated such that the oxidation reactions, such as the lignin oxidation reactions, are carried out up to 200 min, such as 10-200 min, 50-150 min, 60-120 min, 80-120 min or about 100 min. In an embodiment, when more than one reactor is used, the time refers to the total time the oxidative reaction conditions are maintained, including feeding of the oxidative agent into the reactor(s).

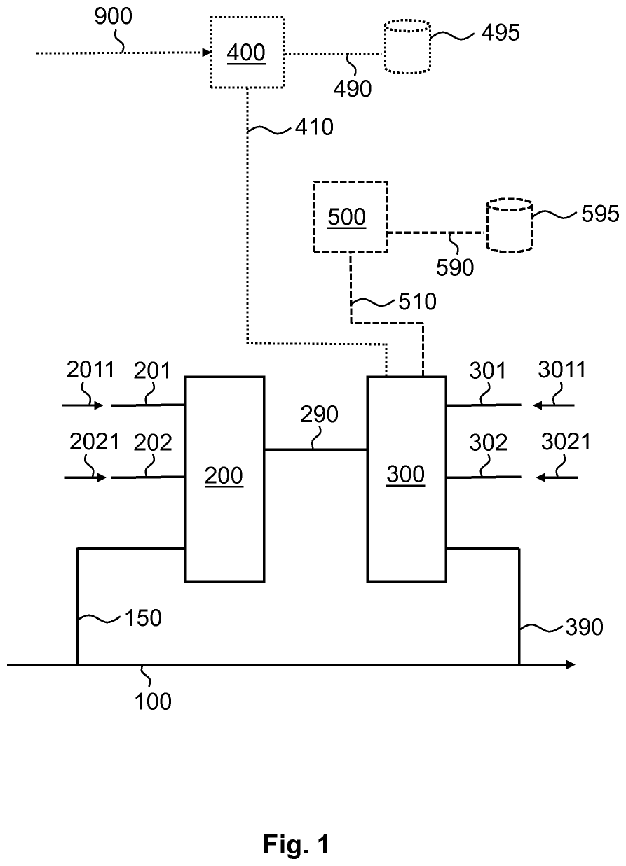

When using the two-reactor configuration illustrated in FIG. 1, the total residence time of the feedstock in the two reactors is preferably not more than 1000 min.

In an embodiment the residence time of the feedstock in the preconditioning reactor is up to 1000 min, such as 30-100 min, or 500-1000 min. These residence times are preferable when the preconditioning comprises oxidative treatment.

In an embodiment the residence time in the second reactor is up to 1000 min, such as 50-1000 min, or 500-1000 min.

In an embodiment non-condensable gases (NCGs) in the reactor(s) are removed by a non-condensable gas handling system in fluid communication with the reactor(s).

In an embodiment of the two-step process, the preconditioning reactor is operated in conditions comprising a temperature of about 90° C., pH in the range 11-12, a pressure in the range 4-8 bar, and by using air or oxygen as the oxidative agent for up to 100 min.

In an embodiment of the two-step process, the second reactor is operated in conditions comprising a temperature of about 90° C., pH in the range 11-12, a pressure in the range 4-8 bar, and by using oxygen or ozone as the oxidative agent for up to 100 min.

In an embodiment the feedstock contains softwood black liquor. Softwood black liquor processing by the present process may comprise a step of controlling pH by adding alkali, such as NaOH. When using softwood black liquor as the feedstock, the operating temperature during oxidative reactions is preferably about 90° C. and optionally the pressure is about 4-8 bar. The pressure can be controlled by using a gaseous oxidative agent, such as oxygen, which is fed into the second reactor to maintain the desired pressure. A higher pressure may be used to increase the production of methanol.

In a preferable embodiment softwood black liquor as the feedstock is processed at about 90° C. and at a pressure of about 8 bar without pH control by additional alkali.

Even 23 kg/Adt of additional methanol could be produced when using softwood black liquor as the feedstock in the present process. Thereby, the total methanol production from black liquor can be increased to over 30 kg/Adt of pulp. Without the present process, the total methanol production of previous processes is approximately 6-10 kg/Adt pulp.

In a preferable embodiment the feedstock contains hardwood black liquor. Hardwood black liquor is preferable because it produces a high yield of methanol. When using hardwood black liquor as the feedstock, the operating temperature is preferably about 90° C. and optionally the pressure is about 8 bar. The pressure can be controlled by using a gaseous oxidative agent, such as oxygen, which is fed into the second reactor to maintain the desired pressure.

In an embodiment the oxidative agent is gaseous.

In an embodiment a gaseous oxidative agent is used to maintain the pressure inside the rector at the desired level. For example, a pressure of 8 bars can be achieved by feeding to the reactor oxygen such that the partial pressure of oxygen inside the reactor is 8 bars.

In an embodiment the oxidative agent is fed into the reactor through a nozzle or an inlet port arranged inside the reactor, or on a wall, bottom plate, and/or top plate of the reactor.

In another embodiment the oxidative agent is mixed in a chemical dynamic mixer or in mixing circulation.

In an embodiment the reactor is a stirred tank reactor, in continuous stirred tank reactor or in plug flow reactor.

In an embodiment the oxidized feedstock is transferred back to black liquor line of the pulp mill after the oxidative treatment. Because the oxidized feedstock fed into the black liquor line of the pulp mill contains an increased amount of methanol, the present process enhances production of methanol in pulp mills, and more methanol can be recovered from black liquor.

The oxidized black liquor product can be transferred back to evaporation plant, where methanol is recovered by using a stripping column and a methanol liquefaction unit from foul condensate. This can be optionally followed by methanol purification. Methanol purification can be carried out for example by distillation, or in a purification plant comprising a series of distillation columns. In another embodiment methanol can be purified by evaporating in an evaporation plant as a foul condensate, from which methanol can be separated by a foul condensate treatment consisting of a foul condensate stripper and an optional methanol liquefication unit.

Another advantage of the present process is that the existing methanol recovery means, such as methanol evaporation, condensing and purification units present at a pulp mill, can be utilized to recover the methanol produced with the present process. The methanol produced in the second reactor can thus be recovered with existing equipment by feeding them with the fluids produced in the second reactor.

Alternatively or additionally, the methanol produced with the present process is recovered near the oxidation reactor. In this embodiment the reactor is preferably operated such that at least part of the methanol is in a vapor form, and methanol can thus be recovered in a liquefication unit directly in fluid connection with the reactor. In one embodiment the oxidation reaction and the removal of methanol happen simultaneously. When the liquefication unit is directly in fluid connection with the reactor, at least part of the methanol present and formed in the oxidized feedstock is recovered before the oxidized feedstock is transferred back to the black liquor circulation of a pulp mill.

In a further alternative or additional embodiment, methanol is recovered in a stripper off gas (SOG) line in fluid connection with the reactor. In an embodiment the SOG line is directly in fluid connection with the reactor.

Alternatively or additionally, gases from the reactor are directed to a SOG line which is in fluid connection to at least one further source of gases produced in a pulp mill. In this configuration the methanol containing gases produced in the reactor can be processed with equipment present in pulp mills, such as in black liquor stripper.

Because methanol boils at low temperature, most of the methanol can be extracted by using a simple stripping and/or distillation of gases evaporated from the oxidized filtrate. This can be done in black liquor evaporation plant, where methanol is transferred to, and recovered from, the foul condensate. The condensed methanol can then be transferred to stripping and methanol liquefication unit.

In an embodiment the present process is a continuous process. The continuous process can be integrated into the process of the pulp mill.

An example embodiment disclosing certain parts of a system configured to carry out the present process is illustrated in FIG. 1 showing a two-reactor system, in which the preconditioning reactor 200 is connected to a BL line 100 of a pulp mill via a reactor inlet line 150, which can be configured to feed BL into the reactor 200.

The preconditioning reactor 200 is connected to an oxidant feed inlet line 201, which can be configured to feed the optional oxidative agent into the preconditioning reactor 200 in a direction shown by the arrow 2011. Alkali can be fed to the preconditioning reactor through an alkali feed inlet line 202 in the direction shown by the arrow 2021. In case acid or other chemical is fed into the preconditioning reactor to adjust pH, said agents can be fed into the preconditioning reactor through the inlet 201, 202, or through another first reactor inlet not shown in FIG. 1.

A first reactor outlet line 290 connects the preconditioning reactor to the second reactor 300 and feeds desulphurized feedstock to the second reactor 300 in which the lignin oxidation takes place. In a preferable embodiment, sulphurous compounds that are reactive to oxygen or ozone are at least partially removed in the preconditioning reactor, and the feedstock entering the second reactor does not contain a significant amount of such compounds. Therefore, in the second reactor the oxidative agent oxidizes primarily methoxyl groups of lignin.

To the second reactor 300 is connected an oxidant feed inlet line 301, which can be configured to feed the oxidant into the second reactor in a direction shown by the arrow 3011. Alkali can be fed to the second reactor through an alkali feed inlet line 302 in the direction shown by the arrow 3021. In case acid or other chemical is fed into the second reactor to adjust pH, said agents can be fed into the second reactor through the inlet 301, 302, or through other second reactor inlets not shown in FIG. 1.

A second reactor outlet line 390 is connected to the BL line 100 in a position downstream of the position in which the reactor inlet line 150 connects to the BL line 100. Oxidized filtrate can be removed from the reactor through the line 390 into the black liquor circulation.

Methanol produced in the oxidation process is dissolved into the oxidized black liquor inside the second reactor. A minor amount of methanol may form in the first reactor in case excess oxygen or air is used and all reactive sulfide compounds are oxidized in the preconditioning reactor. The methanol can be recovered from the oxidized feedstock, which can be fed into the BL line 100 by using equipment present in a pulp mill and which is used for removing methanol from the BL or from other methanol containing feedstocks. In such an embodiment the units 400 and/or 500 shown in dotted lines are optional, and not necessarily present in the system.

In an alternative or additional embodiment, FIG. 1 shows a liquefication unit 500 and a stripper off gas (SOG) line 400 that can be used to remove methanol directly from the vapor formed inside the reactor 300. In the embodiment shown in FIG. 1 these units are directly connected to the second reactor 300. The reactor gas outlet 510 is in fluid connection to the liquefication unit 500 and it conducts gases, including methanol in gas and/or vapor phase, from inside the second reactor 300 into the liquefication unit 500. From the liquefication unit 500 the methanol condensed from gas is conducted through a liquefication unit outlet 590 to a methanol storage 595. The methanol storage 595 can also be configured to receive methanol from other methanol recovery units of the pulp mill, such as from a unit which recovers methanol from BL line 100 (not shown in FIG. 1).

In another alternative or additional embodiment, FIG. 1 shows a SOG line 400 to which a reactor gas outlet 410 is in fluid connection to conduct gases, including methanol in gas and/or vapor phase, from the second reactor to the SOG line 400. From the SOG line 400 the methanol condensed from gas is conducted through an outlet 490 to a methanol storage 495. The methanol storage 495 can be configured to receive methanol from other methanol recovery units of the pulp mill, such as from a unit which recovers methanol from the BL line 100 (not shown). The SOG line 400 can also be configured to receive gas from other sources of the pulp mill as shown by the inlet line 900.

The inlet and outlet lines that are configured to transfer material and are shown in FIG. 1, can be equipped with one or more valve and one or more pump to allow better control of the process, and to ensure efficient transfer of gaseous and liquid steams in different parts of the system. Sampling points can be arranged in pipes or vessels of the system to allow analysis of the material in the process, as well as other process parameters.

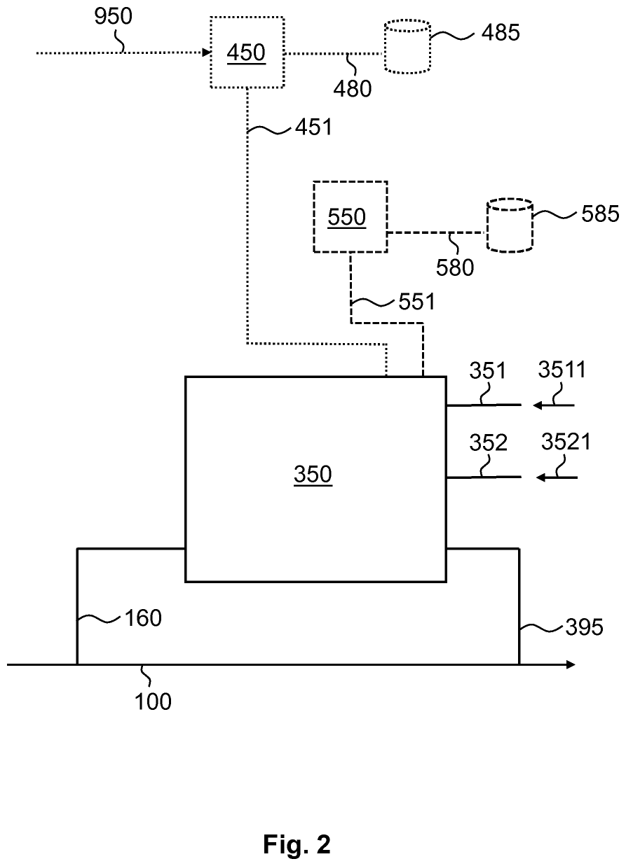

An example embodiment disclosing certain parts of a system configured to carry out the present process is illustrated in FIG. 2 showing a one-reactor system, in which the second reactor 350 is connected to a BL line 100 of a pulp mill via a reactor inlet line 160, which can be configured to feed BL into the second reactor 350.

To the second reactor 350 is connected an oxidant feed inlet line 351, which can be configured to feed the oxidant into the second reactor in a direction shown by the arrow 3511. Alkali can be fed to the second reactor through an alkali feed inlet line 352 in the direction shown by the arrow 3521. In case acid or other chemical is fed into the second reactor to adjust pH, said agents can be fed into the second reactor through the inlet 351, 352, or through another first reactor inlet not shown in FIG. 2.

A reactor outlet line 395 is connected to the BL line 100 in a position downstream of the position in which the reactor inlet line 160 connects to the BL line 100. Oxidized filtrate can be removed from the second reactor through the line 395 into the black liquor circulation.

Methanol can be recovered from the oxidized feedstock, which can be fed into the BL line 100 by using equipment present in a pulp mill and which is used for removing methanol from the BL or from other methanol containing feedstocks. In such an embodiment the units 450 and/or 550 shown in dotted lines in FIG. 2 are not necessarily present in the system.

In an alternative or additional embodiment, FIG. 2 shows a liquefication unit 550 and a SOG line 450 that can be used to remove methanol directly from the vapor formed inside the second reactor 350. In the embodiment shown in FIG. 2 these units are directly connected to the second reactor 350. The reactor gas outlet 551 is in fluid connection to the liquefication unit 550 and it conducts gases, including methanol in gas and/or vapor phase, from the second reactor 350 into the liquefication unit 550. From the liquefication unit 550 the methanol condensed from gas is conducted through a liquefication unit outlet 580 to a methanol storage 585. The methanol storage 585 can also be configured to receive methanol from other methanol recovery units of the pulp mill, such as from a unit which recovers methanol from BL line 100 (not shown in FIG. 2).

In another alternative or additional embodiment, FIG. 2 shows a SOG line 450 to which a reactor gas outlet 451 is in fluid connection to conducts gases, including methanol in gas phase, from the second reactor to the SOg line 450. From the SOG line 450 the methanol condensed from gas is conducted through an outlet 480 to a methanol storage 485. The methanol storage 485 can be configured to receive methanol from other methanol recovery units of the pulp mill, such as from a unit which recovers methanol from the BL line 100 (not shown). The methanol storage 485 can also be configured to receive gas from other sources of the pulp mill as shown by the inlet line 950.

The inlet and outlet lines that are configured to transfer material and are shown in FIG. 2 can be equipped with one or more valve and one or more pump to allow better control of the process, and to ensure efficient transfer of gaseous and liquid phases in different parts of the system. Sampling points can be arranged in pipes or vessels of the system to allow analysis of the material in the process, as well as other process parameters.

Heating and/or cooling means can be arranged in the reactor(s) to control the operating temperature of the reactor.

### EXAMPLES

### Example 1. Methanol Production from Softwood Black Liquor

Softwood black liquor as the feedstock was used to study the effect of different operating conditions in the present process. The operating conditions and their effect on the methanol production are shown in Table 1.

In the experiments one 8 liter batch reactor was used. Pure O₂ was used as the oxidative agent. Operational pressure was 4-8 bar(g). Pressure was controlled with O₂ flow. Temperature was 80-100° C. In some tests pH was controlled by adding NaOH. Residence times were max 300 min. MeOH was measured directly from BL with GC and also by analyzing changes in lignin methoxyl group contents with P-NMR

The following abbreviations are used: DM: dry matter in mass-%; tds: total dissolved solid.

TABLE 1
Methanol production from softwood black liquor.

| DM | Lignin | p(O₂) | Avg T | pH | t | MeOH | MeOH |
|---|---|---|---|---|---|---|---|
| (initial) | (initial) | bar | ° C. | control | min | kg/tds | kg/ADT |
| 30% | 10% | 4 | 80 | 0 | 95 | 6.3 | 11.6 |
| 42% | 13% | 4 | 80 | 0 | 95 | 3.5 | 6.5 |
| 42% | 13% | 4 | 80 | 1 | 95 | 5.6 | 10.3 |
| 30% | 10% | 4 | 90 | 1 | 95 | 7.4 | 13.7 |
| 30% | 10% | 4 | 100 | 1 | 95 | 6.2 | 11.5 |
| 30% | 10% | 4 | 90 | 0 | 200 | 11.3 | 20.8 |
| 30% | 10% | 8 | 90 | 0 | 200 | 12.4 | 22.9 |

As the results show, lignin present in softwood black liquor was effectively converted to methanol by the present process.

Effective production of methanol from lignin was achieved in all conditions shown in Table 1. Each of the parameters increased pressure, increased pH, and increased reaction time individually increased methanol production. Methanol production was increased in the experiments even further when increasing pressure to 8 bar and when using a temperature of 90° C.

The results show that pH control is not necessary to produce methanol, and high methanol production can be reached without alkali addition. In addition, lower DS %-content provided better results for both HW black liquor and SW black liquor.

### Example 2. Methanol Production from Hardwood Black Liquor

Hardwood black liquor as the feedstock was used to study the effect of different operating conditions in the present process. The operating conditions and their effect on the methanol production are shown in Table 2.

In the experiments one 8 liters batch reactor was used. Pure O₂ was used as the oxidative agent. Operational pressure was 4-8 bar(g). Pressure was controlled with O₂ flow. Temperature was 90° C. pH was not controlled. Reaction time was 200 minutes. MeOH was measured directly from BL with GC and also by analyzing changes in lignin methoxyl group contents with P-NMR

TABLE 2
Methanol production from hardwood black liquor.

| DM | Lignin | p(O₂) | Avg T | pH | | MeOH | kg/ADT |
|---|---|---|---|---|---|---|---|
| (initial) | (initial) | bar | ° C. | control | min t | kg/tds | MeOH |
| 30% | 9% | 8 | 90 | 0 | 200 | 27.1 | 37.94 |
| 44% | 12% | 8 | 90 | 0 | 200 | 27.7 | 38.78 |
| 20% | 6% | 8 | 90 | 0 | 200 | 28.2 | 39.48 |
| 30% | 9% | 4 | 90 | 0 | 200 | 20.1 | 28.14 |

As the results show, lignin present in hardwood black liquor was effectively converted to methanol by the present process. The methanol yield with hardwood black liquor is larger than when using softwood.

Effective production of methanol from lignin was achieved in all conditions shown in Table 2. Increased pressure increased methanol production.

Further, without pH control significant methanol production was observed. With higher pressure, the methanol production yield was higher.

The foregoing description has provided by way of non-limiting examples of particular implementations and embodiments a full and informative description of the best mode presently contemplated by the inventors for carrying out the invention. It is however clear to a person skilled in the art that the invention is not restricted to details of the embodiments presented in the foregoing, but that it can be implemented in other embodiments using equivalent means or in different combinations of embodiments without deviating from the characteristics of the invention.

Furthermore, some of the features of the afore-disclosed example embodiments may be used to advantage without the corresponding use of other features. As such, the foregoing description shall be considered as merely illustrative of the principles of the present invention, and not in limitation thereof. Hence, the scope of the invention is only restricted by the appended patent claims.

1. A process for producing methanol comprising:
providing a feedstock comprising black liquor;
preconditioning the feedstock in a preconditioning reactor operated at a temperature in a range of 80° C. to 160° C. to provide preconditioned feedstock;
transferring the feedstock or the preconditioned feedstock to a second reactor; and
feeding into the second reactor a first oxidative agent to produce a reaction mixture to oxidize methoxyl groups of lignin present in the black liquor into methanol.

2. The process of claim 1, wherein the preconditioning is performed in alkaline conditions.

3. The process of claim 1, wherein the preconditioning includes feeding a second oxidative agent into the preconditioning reactor, wherein the second oxidative agent is selected from at least one of: air, oxygen, and an air water mixture.

4. The process of claim 1, wherein the first oxidative agent is selected from one or more of: oxygen, ozone, air, or a mixture of oxygen, ozone and/or air.

5. The process of claim 1, wherein the first oxidative agent is fed into first nozzle or first inlet port of the preconditioning reactor and/or into a second nozzle or second inlet port of the second reactor.

6. The process of claim 1, wherein the reaction mixture has a pH and the process further comprises adjusting the pH of the reaction mixture to be within a range of 8 to 14.

7. The process of claim 1, wherein an operating temperature within the preconditioning reactor and/or the second reactor is in a range of 85° C. to 140° C.

8. The process of claim 1, wherein an operating pressure within the preconditioning reactor and/or the second reactor is in a range of 2 bar(g) to 16 bar(g).

9. The process of claim 1, wherein the feedstock comprises at least one of softwood black liquor or hardwood black liquor.

10. The process of claim 1, wherein the feedstock contains sulfuric compounds.

11. The process of claim 1, further comprising transferring the feedstock from the second reactor to black liquor circulation of a pulp mill.

12. The process of claim 1, wherein the methanol is at least partially in gaseous form in the second reactor, and
the process further comprises transferring the methanol from the second reactor to a liquefication unit or to a stripper off-gas line.

13. (canceled)

14. A process including:
introducing a feed stock including black liquor and lignin in a preconditioning reactor;
processing, in the preconditioning reactor, the feedstock at a temperature in a range of 80° C. to 160° C.;
transferring the feedstock from the preconditioning reactor to a second reactor; and
reacting, in the second reactor, the feedstock with a first oxidative agent under alkaline conditions to oxidize methoxyl groups of the lignin in the black liquor to form methanol.

15. The process of claim 14, further comprising feeding a second oxidative agent into the preconditioning reactor, and
reacting the second oxidative agent with the feedstock in the preconditioning reactor, wherein the second oxidative agent includes at least one of: air or oxygen.

16. The process of claim 14, wherein the first oxidative agent includes one or more of: oxygen, ozone or air.

17. The process of claim 14, further comprising adjusting a pH of the feedstock in the second reactor to be within a range of 8 to 14.

18. The process of claim 14, wherein an operating pressure within the preconditioning reactor and/or the second reactor is in a range of 2 bar(g) to 16 bar(g).

19. The process of claim 14, wherein the feedstock contains sulfuric compounds and the process further comprises separating from the feedstock in the preconditioning reactor vapors produced from the sulfuric compounds before the feedstock is transferred to the second reactor.

20. The process of claim 14, wherein the methanol is at least partially a vapor and the process further comprises transferring the methanol from the second reactor to a liquefication unit or to a stripper off-gas line.
